(19) United States
(12) Patent Application Publication
(10) Pub. No.: US 2004/0071707 A1
(43) Pub. Date: Apr. 15, 2004

(54) Modulation of cell growth

(76) Inventors: Veronica A. Carroll, London (GB); Adrian L. Harris, Oxford (GB); Roy Bicknell, Oxford (GB)

Correspondence Address: Nixon & Vanderhye, 8th Floor, 1100 North Glebe Road, Arlington, VA, 22201-4714, US

(21) Appl. No.: 10/432,842

(22) PCT Filed: Nov. 28, 2001
(86) PCT No.: PCT/GB01/05244

(30) Foreign Application Priority Data
Nov. 28, 2000 (GB) 0029001.5

(51) Int. Cl. A61K 39/395; G01N 33/53; G01N 33/567; C07K 16/40

(52) U.S. Cl. 424/146.1; 435/7.2; 530/388.26

(57) ABSTRACT

A compound which inhibits binding of kringle 2 domain of tissue plasminogen activator (tPA) to endothelial cells is provided for use in stimulating proliferation of endothelial cells. A kringle 2 domain of tPA may also be used to reduce endothelial cell proliferation or to induce cell death.

### FIELD OF THE INVENTION

The present invention relates to use of agents which inhibit binding of kringle 2 domain of tissue plasminogen activator to endothelial cells for use in therapy. The compounds may be used alone for stimulating proliferation of endothelial cells. Alternatively such agents may be used in combination with an agent which prevents binding of the finger domain for reducing endothelial cell proliferation. Additionally, a kringle 2 domain may be used itself for reducing endothelial cell proliferation.

### BACKGROUND TO THE INVENTION

Tissue plasminogen activator (tPA) is primarily responsible for the conversion of plasminogen into plasmin and subsequent removal of fibrin from the vasculature during clot dissolution and wound healing. In addition, fibrinolytic activity has been associated with a variety of other physiological processes such as ovulation, embryogenesis, fertilization and memory. Less attention has been focused on the role of tPA in tumour progression as compared with the urokinase plasminogen activator (uPA) dependent pathway of plasminogen activation. While uPA is widely accepted to be the plasminogen activator (PA) expressed by tumor cells during invasion and metastasis dissemination, t-PA has also been implicated in tumor cell invasion and may also promote metastasis of intraocular melanoma. In acute nonlymphocytic leukemias poor outcome correlated with high tPA levels. In contrast, others have shown that elevated t-PA determined in cytosol extracts may be a good prognostic marker for some types of cancer.

TPA is synthesized and expressed by endothelial cells and has been localized to the vasa vasorium of normal vessels. It is a multi-domain protein that consists of a finger domain, an epidermal growth factor (EGF)-like domain, two kringle modules with high homology to the kringle domains contained in plasminogen and a protease domain.

### SUMMARY OF THE INVENTION

The separate domains of tPA have been found to influence endothelial cell growth independently of the catalytic activity of tPA. We show that the kringle 2 domain of tPA is growth inhibitory for endothelial cells, and that an inhibitor of kringle 2 can promote proliferation. This proliferative activity is mediated through the finger domain and can be inhibited using an inhibitor of the finger domain. The presence of both proliferative and antiproliferative domains in tPA as well as the close proximity of tPA to the endothelium suggests that tPA may play a role in angiogenesis in vivo during wound healing in addition, manipulation of the tPA pathway may be a potential new target for both stimulating and inhibiting angiogenesis in vivo.

In accordance with one aspect of the present invention there is provided an agent which inhibits binding of kringle 2 domain of tissue plasminogen activator (tPA) to endothelial cells for use in stimulating proliferation of endothelial cells. Preferably, the compound comprises an anti-kringle 2 domain antibody.

The invention also provides, in combination, (a) an agent which inhibits binding of kringle 2, and (b) tPA or a fragment thereof including at least a finger domain, for simultaneous or sequential administration for use in stimulating proliferation of endothelial cells.

The proliferative agents or combinations of the present invention may be used in wound healing, coronary artery disease and critical limb ischaemia.

In another aspect, the invention provides a method for the identification of a substance useful for stimulating proliferation of endothelial cells, comprising incubating a kringle 2 domain of tPA with an endothelial cell membrane in the presence of a test substance, monitoring for binding of the kringle 2 domain to the endothelial cell membrane, and determining thereby whether the test substance is useful in blocking kringle 2 binding to endothelial cells and subsequent proliferation of endothelial cells.

In an alternative aspect the invention provides a kringle 2 domain of tPA, for use in inducing cell death or reducing proliferation of endothelial cells.

The invention also provides, in combination an agent which inhibits binding of kringle 2 domain and an agent which inhibits binding of the finger domain of tPA to endothelial cells, for simultaneous or sequential administration for use in inducing cell death or reducing proliferation of endothelial cells.

The anti proliferative agent or combination of the present invention may be used in the treatment of solid tumours, rheumatoid arthritis and diabetic retinopathy.

### IN THE FIGURES

FIG. 1. Antibody mediated blocking of the kringle 2 domain of tPA promotes HUVEC growth.

Cell proliferation assays. (A). HUVEC cultures were incubated with a panel of monoclonal anti-tPA antibodies at the concentrations indicated for four days according to Materials and Methods. Anti-finger/EGF (3VPA and 6VPA), anti-kringle 1 (PAM-2), anti-kringle 2 (7VPA) and anti-protease (374B and ESP2). Results are expressed as a percentage of control wells containing M199 with 2% FBS which were designated as 100% cell growth. Values represent mean±SE of at least two experiments. (B). Dose dependent HUVEC proliferation with an anti-kringle 2 antibody (7VPA). Data represent mean±SE of at least three independent experiments with two batches of HUVEC each pooled from ten donors. *p<0.05, **p<0.005 t-test. (7VPA). (C) Addition of D-mannitol to HUVEC cultures. Values represent mean±SE of three experiments. (D) HUVEC were incubated with 10 ng/mL of either rsctPA or alatPA, a catalytically inactive mutant. In addition, VEGF was added as a positive control at 10 ng/mL. Results are mean±SE of duplicate determinations.

FIG. 2. Anti-kringle 2 mediated HUVEC proliferation is dependent on the finger domain of tPA.

(A). HUVEC were stimulated to proliferate with either 700 nM or 1400 nM anti-kringle 2 antibody (7VPA) with the simultaneous addition of an anti-finger/EGF antibody (3VPA) at the concentrations indicated. 100% cell growth represents absorbance of control wells in which cells were grown with 7VPA alone. Data represent mean of two independent experiments. (B). HUVEC were grown as in A with 700 nM anti-kringle 2 antibody (7VPA) with the simultaneous addition of 700 nM anti-kringle 1 (PAM-2) antibody, 700 nM anti-protease domain antibody (374B) or with a protease inhibitor aprotinin at the concentrations indicated. Results express mean±SE of duplicate determinations. Control wells represent cell growth in which wells contained anti-kringle 2 antibody (7VPA) alone (C). HUVEC were incubated with a synthetic peptide of tPA (tPA I) corresponding to residues 7-17 of the finger domain in the absence (control) or presence of either 700 nM anti-kringle 2 antibodies 7VPA or ESP1 for four days. Results represent average of two independent experiments. (D). HUVEC were incubated with a second peptide of tPA (tPA II) corresponding to residues 15-26 of the finger domain in the absence (control) or presence of 700 nM anti-kringle 2 (7VPA). Values represent mean±SE of two experiments. (E). Viability of HUVEC 24 h after the addition of tPA I in the absence (control) or presence of 7VPA at the concentrations indicated. Data are mean±SE of one representative experiment.

FIG. 3. Anti-kringle 2 stimulated HUVEC growth is dependent on PI3-kinase.

(A). HUVEC were stimulated in the presence of either 700 nM anti-kringle 2 (7VPA) or 0.4 nM (10 ng/mL) VEGF with the simultaneous addition of a PI3-kinase inhibitor LY294002 at the concentrations indicated. Results are mean±SE of three independent experiments. (B) Effect of LY294002 alone on HUVEC cultures. (C). HUVEC cultures were stimulated with 700 nM anti-kringle 2 together with a PKA inhibitor Rp-cAMPS or an EGF receptor tyrosine kinase inhibitor Lavendustin A at the concentrations indicated. Data are mean±SE of two dependent experiments.

FIG. 4. Effect of exogenous kringle 2 of tPA on HUVEC and HVSMC growth.

(A and B). HUVEC or HVSMC cultures were incubated with increasing concentrations of PMSF inactivated K2P (K2P-PMSF) or control treated K2P either in the absence (A) or presence (B) of 10 ng/mL bFGF at the concentrations indicated. Angiostatin was included as a positive control for inhibition of bFGF mediated cell growth. HUVEC results represent mean of at least three independent experiments with three different batches of HUVEC. Experiments with HVSMC were repeated twice. (C and D). HUVEC were treated with 100 μg/mL K2P-PMSF and increasing concentrations of either anti-kringle 2 antibody, 7VPA (C) or rsctPA (D) for four days as indicated. Control wells contained M199 and 2% FBS alone. Data represent mean±SE of duplicate experiments with two different batches of HUVEC.

FIG. 5. Effect of elastase cleaved kringle 2 on endothelial cell growth.

Purified K2 was incubated with HUVEC for 4 days either alone or in the presence of 10 ng/mL bFGF or VEGF as indicated. Values represent mean±SD of triplicate determinations.

### DETAILED DESCRIPTION

The present invention provides an agent which inhibits the binding of kringle 2 domain of tissue plasminogen activator (tPA) to endothelial cells for use in therapy. The sequence of mature tPA is given in SEQ ID NO: 1. The kringle 2 domain comprises residues 180-261 of SEQ ID NO: 1. A nucleotide sequence encoding the kringle 2 domain is given in SEQ ID NO: 2.

Inhibitors useful in accordance with the present invention can be identified by monitoring the binding of the kringle 2 domain to endothelial cells or endothelial cell membranes in the presence of a test substance. Suitable binding assays include providing a labeled kringle 2 domain and incubating the kringle 2 domain with endothelial cell membranes, for example, using endothelial cell mono layers or endothelial cells in suspension. The kringle 2 domain may be labeled by any suitable label such as a radiolabel, fluorescent label or colorimetric label.

Preferably, full length tPA is not used in the assessment of kringle 2 binding to endothelial cells. Other regions of tPA are also involved in endothelial cell binding such as the finger domain which may interfere in interpretation of the results. The finger domain comprises amino acid residues 4 to 46 of SEQ ID NO: 1. Thus, preferably, a fragment of tPA incorporating a kringle 2 domain is used and in particular, a truncated tPA molecule in which the finger domain or the region of the finger domain associated with endothelial cell binding has been deleted. Assays may readily be carried out using an isolated kringle 2 domain such as a peptide comprising residues 180-261 of SEQ ID NO: 1. In a preferred assay, labeled kringle 2 is added to endothelial cells in the presence of a test substance. The bound fraction can be determined, for example, by subtracting the amount of unbound label from the total amount added to the cells. The amount of the bound fraction decreases in the presence of specific inhibitors of kringle 2 binding. Binding assays are preferably carried out on intact cells.

One aspect of the present invention provides an assay for the identification of proliferative agents by identifying agents which inhibit kringle 2 binding to endothelial cell. Such assays are described in more detail below. Agents identified in accordance with the present invention are preferably formulated with a pharmaceutically acceptable carrier for subsequent use.

Inhibitors of kringle 2-mediated tPA binding to endothelial cells are now shown to induce endothelial cell proliferation. Thus, an agent which inhibits the binding of kringle 2 domain to endothelial cells is preferably used in a method of therapy where it is desired to stimulate endothelial cell proliferation. In particular, the agents of the present invention may be useful in wound healing, coronary artery disease and critical limb ischaemia. The proliferative activity is mediated through other domains of tPA which also bind to endothelial cells and in particular is mediated through binding of the finger domain.

In one aspect of the invention, an inhibitor of kringle 2 binding domain is administered together with fill length tPA or a variant thereof which maintains the proliferative activity of tPA in the presence of an inhibitor of kringle 2 domain binding. In accordance with this aspect, tPA and the kringle 2 binding inhibitor are administered in an amount such that the inhibitor of kringle 2 domain binding blocks binding of tPA via the kringle 2 domain but allows for other tPA binding such that proliferation of the endothelial cells is induced. In a preferred aspect, the variant of tPA is a fragment thereof comprising at least the finger domain. Suitable variants or fragments of tPA may be identified by monitoring for endothelial cell proliferation in the presence of an inhibitor of kringle 2 binding to endothelial cells and the variant or fragment of tPA.

In an alternative aspect of the invention, a kringle 2 domain of tPA may be used alone for inducing cell death or reducing proliferation of endothelial cells. Thus, a kringle 2 domain or fragment or variant thereof may be administered to induce cell death or reduce proliferation of endothelial cells. In particular, such an antiproliferative agent may be used in therapy where a reduction in proliferation of endothelial cells is required such as the treatment of solid tumours, rheumatoid arthritis or diabetic retinopathy. Preferably, the kringle 2 domain for use in accordance with this aspect of the invention comprises the kringle 2 domain comprising residues 180-261 of SEQ ID NO: 1 Alternatively, the kringle 2 domain may comprise a fragment or variant of the sequence sufficient to induce cell death or reduce proliferation of endothelial cells. Alternatively, a fragment of tPA comprising at least a kringle 2 domain such as a fragment comprising a kringle 2 domain and the protease domain which may or may not be inactivated, may be used in accordance with this aspect of the invention.

A kringle 2 domain for use in the assays in accordance with the present invention or for administration may comprise naturally occurring kringle 2 of tPA having the sequence of residues 180-261 of SEQ ID NO: 1. Alternatively, a variant kringle 2 domain may be used. Such variants preferably maintain the function of the kringle 2 domain such as kringle 2 binding to endothelial cells or the ability to reduce proliferation of endothelial cells. The effect on endothelial cell proliferation can be readily monitored, for example, using a suitable in vitro assay. Similarly, tPA for use in accordance with the present invention may comprise naturally occurring tPA having the sequence of SEQ ID NO: 1 or a fragment or variant thereof which maintains a function of tPA and in particular is able to stimulate proliferation of endothelial cells, in particular, in the presence of an inhibitor of kringle 2 domain binding. In particular a fragment comprises at least the finger domain of tPA.

Typically, polypeptides with more than about 65% identity preferably at least 80% or at least 90% and particularly preferably at least 95% at least 97% or at least 99% identity, with the amino acid sequences of SEQ ID NO: 1, or a fragment thereof, such as the kringle 2 domain, are considered as variants of the proteins. Such variants may include naturally occurring variants such as allelic variants and the deletion, modification or addition of single amino acids or groups of amino acids within the protein sequence. In particular, one skilled in the art is readily able to identify kringle 2 domains from other tPA molecules which differ from the specific sequence set out in SEQ ID NO: 1. In particular, a variant tPA molecule may be aligned with the tPA sequence of SEQ ID NO: 1 and, based on the closest sequence alignment, a kringle 2 domain of such a variant sequence can be identified.

Amino acid substitutions may be made, for example from 1, 2 or 3 to 10, 20 or 30 substitutions. The modified polypeptide generally retains an activity of tPA or kringle 2 activity. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar13 uncharged | C S T M |
| | | N Q |
| | Polar13 charged | D E |
| | | K R |
| AROMATIC | | H F W Y |

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for kringle 2 or variant thereof or tPA or variant thereof as well as nucleotide sequences which are complementary thereto for use in accordance with the invention. The nucleotide sequence may be RNA or DNA including genomic DNA, synthetic DNA or cDNA. Preferably the nucleotide sequence is a DNA sequence and most preferably, a cDNA sequence. Nucleotide sequence information for the amino acid sequence of SEQ ID NO: 1 is set out in, for example, Pennica et al. Nature 301, 1983, 214-221. The DNA sequence encoding the kringle 2 domain is given in SEQ ID NO: 2. Such nucleotides can be isolated from human cells or synthesised according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of SEQ ID NO: 1, and in particular of hybridizing under selective conditions to the sequence of SEQ ID NO: 2.

A polynucleotide of the invention can hydridize to SEQ ID NO: 2 or to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and SEQ ID NO: 2 the coding sequence or complement of the coding sequence of SEQ ID NO: 1 is typically at least 10 fold, preferably at least 100 fold, as intense as interactions between other polynucleotides and SEQ ID NO: 2 or the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989). For example, if high stringency is required suitable conditions include from 0.1 to 0.2×SSC at 60° C. up to 65° C. If lower stringency is required suitable conditions include 2×SSC at 60° C.

SEQ ID NO: 2 or the coding sequence of SEQ ID NO: 1 may be modified by nucleotide substitutions, for example from 1, 2 or 3 to 10, 25, 50 or 100 substitutions. SEQ ID NO: 2 or the polynucleotide encoding SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has kringle 2 activity or tPA activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 or of SEQ ID NO: 2 will generally have at least 60%, at least 70%, at least 80%, at least 90%, at least 95%, at least 98% or at least 99% sequence identity to SEQ ID NO: 2 or the coding sequence of SEQ ID NO: 1 over a region of at least 20, preferably at least 30, for instance at least 40, at least 60, more preferably at least 100 contiguous nucleotides or most preferably over the full length of SEQ ID NO: 2 or a polynucleotide encoding SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5737. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence less than about 1, preferably less than about 0.1, more preferably less than about 0.01, and roost preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmtl and adh promoter. Mammalian promoters include the metallotllionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eucaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right. In particular, an antibody to kringle 2 may be used as an inhibitor of kringle 2 binding to endothelial cells and is useful in stimulating proliferation of endothelial cells.

An antibody, or other compound, “specifically binds” to a protein when it binds with preferential or high affinity to the protein for which it is specific but does substantially not bind or binds with only low affinity to other proteins. A variety of protocols for competitive binding or immunoradiometric assays to determine the specific binding capability of an antibody are well known in the art (see for example Maddox er al, J. Exp. Med. 158, 1211-1226, 1993). Such immunoassays typically involve the formation of complexes between the specific protein and its antibody and the measurement, of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I. providing an antibody of the invention;

II. incubating a biological sample with said antibody under conditions which allow for the formation of an antibody-antigen complex; and

III. determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Bar virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Preferably, antibodies for use in accordance with the present invention and in particular for administration are antibodies which recognise kringle 2 and lead to proliferation of endothelial cells. An example of a preferred antibody in accordance with the present invention comprises the antibody referred to herein as 7VPA as described in Wojta et al J. Biol. Chem., 1989, 264 7957-7961.

The invention also provides an assay for identifying test substances which inhibit kringle 2 binding to endothelial cells, or to assays to identify inhibitors of finger domain binding to endothelial cells. Such assays may be carried out by monitoring for binding of labeled kringle 2 or finger domain to endothelial cell monolayers and/or endothelial cells in suspension in the presence of a test substance. The kringle 2 or finger domain can be labelled using any suitable label such as a fluorescent or radio label. Binding can be monitored by measuring the amount of bound label, for example by subtracting the unbound fraction from the total label added, compared to unbound label. Other assays could be carried out to monitor for endothelial cell proliferation.

Suitable test substances which can be tested in the assays of the invention include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition of kringle 2 binding tested individually. Test substances may be used at a concentration of from 1 mM to 1000 μM, preferably from 1 μM to 100 μM, more preferably from 1 μM to 10 μM, preferably, the activity of a test substance is compared to the activity shown by a known inhibitor of kringle 2 binding. A test substance which acts as an inhibitor may produce a 50% inhibition of binding of kringle 2.

The present invention provides compositions useful for stimulating proliferation of endothelial cells by modulating the effect of kringle 2 domain on endothelial cells. Such substances may be used in inducing proliferation of endothelial cells in vitro or in vivo, for example, to stimulate angiogenesis, wound healing, treatment of coronary artery disease, critical limb ischaemia in diabetes, and other conditions where endothelial cell proliferation is desirable.

In an alternative aspect of the present invention, the invention provides either the use of a kringle 2 domain or alternatively an in inhibitor of kringle 2 function together with at inhibitor of the finger domain of tPA to induce cell death or reduce proliferation of endothelial cells. Such compositions are useful in the treatment of cancers, for example to reduce angiogenesis, and in the treatment of rheumatoid arthritis and diabetic retinopathy.

An inhibitor of the finger domain of tPA is preferably an agent which inhibits finger domain binding of tPA. Such an inhibitor may comprise a small fragment of the finger domain itself which will bind to the relevant endothelial cell receptor and thus prevent tPA binding via the finger domain. An example of such a finger domain comprises residues 7-17 of SEQ ID NO: 1, of the finger domain of tPA. Agents which inhibit finger domain activity may be identified, for example, by carrying out competitive binding assays similar to those described above for the kringle 2 domain. Antibodies which inhibit finger domain binding may also be used.

Substances for administration in accordance with the present invention may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes.

A therapeutically effective amount of an inhibitor of kringle 2 binding is administered to a patient. The dose of compositions for administration may be determined according to various parameters, especially according to the substance used; the age, weight and condition of the patient to be treated; the route of administration, and the required regimen. A physician will be able to determine the required route of administration and dosage for any particular patient. A typical daily dose is from about 0.1 to 50 mg per kg of body weight, according to the activity of the specific agent, the age, weight and conditions of the subject to be treated, the type and severity of the disorder and the frequency and route of administration. Preferably, daily dosage levels are from 5 mg to 2 g.

Nucleic acid encoding kringle 2 or a variant thereof may be administered to the mammal. Nucleic acid encoding tPA may also be administered to a mammal together with an inhibitor of kringle 2 domain binding. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid maybe delivered directly across the skin using a nucleic acid delivery device such as particle-mediated gene delivery. The nucleic acid may be so administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle-mediated gene delivery and 10 μg to 1 mg for other routes.

The following Examples illustrate the invention.

Materials and Methods

Cell Culture

Human umbilical vein endothelial cells (HUVEC) were purchased from Clonetics or isolated from umbilical vein cords by mild collagenase treatment as described and cultured in M199 with Earle's salts (Sigma) with 20% heat inactivated fetal bovine serum (Sigma), 100 IU/mL penicillin 100 μg/mL streptomycin (Gibco), 250 ng/mL fungizone (Gibco), 2 mM glutamine (Gibco), 5 IU/mL heparin (Sigma) and 50 μg/mL ECGS (Technoclone, Vienna, Austria). Human vascular smooth muscle cells (HVSMC) were isolated as described.

In Vitro Endothelial Cell Proliferation Assays

3×10³ cells were plated in 96-well plates and allowed to attach overnight. Growth was arrested by incubation with 100 μL reduced growth medium (M199 with 2% FBS) for 24 h. Thereafter anti-tPA antibodies and other reagents were added to wells. K2P (Reteplase), anti-kringle 2 (7VPA) and anti-finger/EGF antibodies (3VPA and 6VPA) were a gift from Technoclone, Vienna, Austria. Anti-kringle 1 (PAM-2), anti-kringle 2 (ESP1) and anti-protease (374B and ESP2) antibodies were purchased from American Diagnostica, Alpha Labs, UK. D-mannitol and LY294002 were obtained from Sigma. VEGF₁₂₁ and bFGF were purchased from R&D Systems Inc., UK. Recombinant single chain tPA (rsctPA) was obtained from Boehringher Ingelheim. The mutant alatPA was kindly provided by Prof. Roger Lijnen (Center for Molecular and Vascular Biology, Leuven, Belgium). Aprotinin was purchased from Boehringer Mannheim. The tPA peptides (tPA I and tPA II) corresponding to residues 7-17 and 15-26 respectively were synthesized by the Clinical Sciences Centre peptide synthesis facility at Hammersmith and the ICRF peptide unit. Rp-cAMPS and Lavendustin A were obtained from Calbiochem, UK. After 4 days of incubation cell number was quantified colorimetrically by the addition of 20 μL CellTiter 96 AQueous One Solution Cell Proliferation Assay (Promega) at 492 nm and 620 nm reference filter. Background absorbance was determined with substrate alone and subtracted from all wells.

HUVEC were plated at 1×10⁵ cells in 12 well plates overnight. Medium was replaced with reduced growth medium overnight followed by addition of anti-K2 antibody. Control wells contained M199 and 2% FBS alone. After 3 days incubation cells were counted in a hemocytometer.

Inactivation of tPA

K2P was inactivated with two round of 2 mM PMSF (Boehrinher Mannheim) as described. Inhibition of catalytic activity was confirmed with Spectrozyme tPA (American Diagnostica) chromogenic substrate.

TPA Assays

TPA protein levels were determined in cell culture supernatant and extracts of EC cultures by ELISA (Technoclone).

Statistics

Statistical analysis was performed with Instat statistical program.

Results

Effect of Anti-Kringle 2 Antibody on Endothelial Cell Proliferation

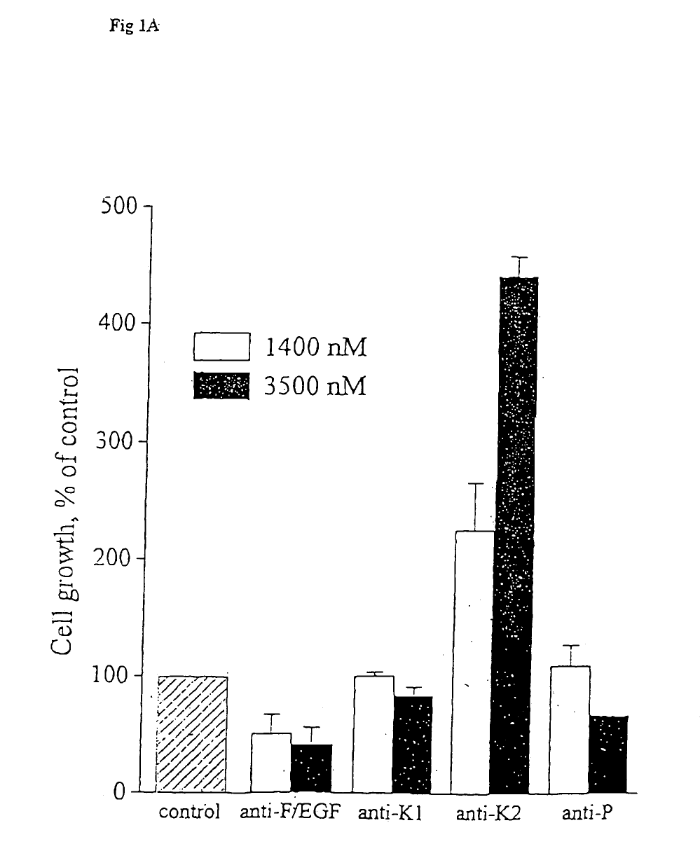

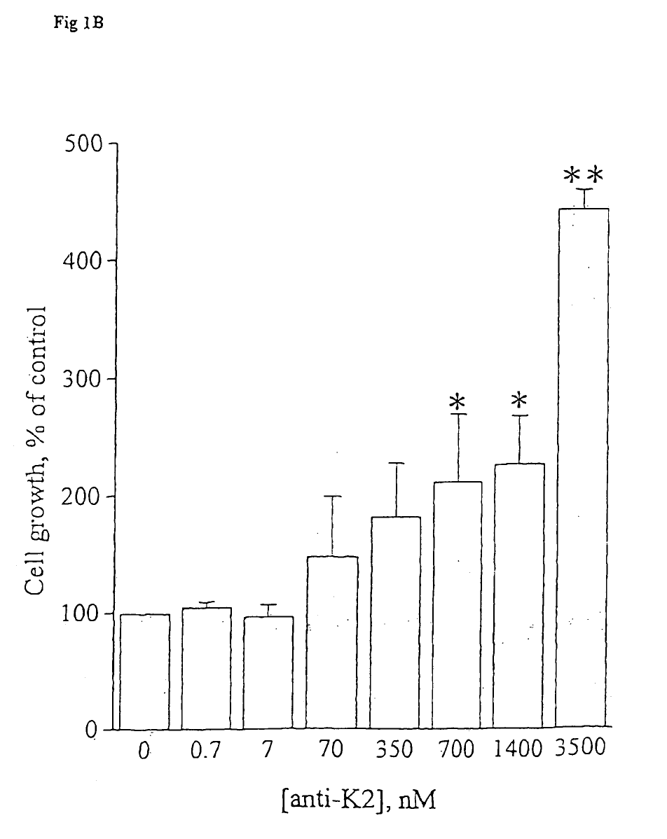

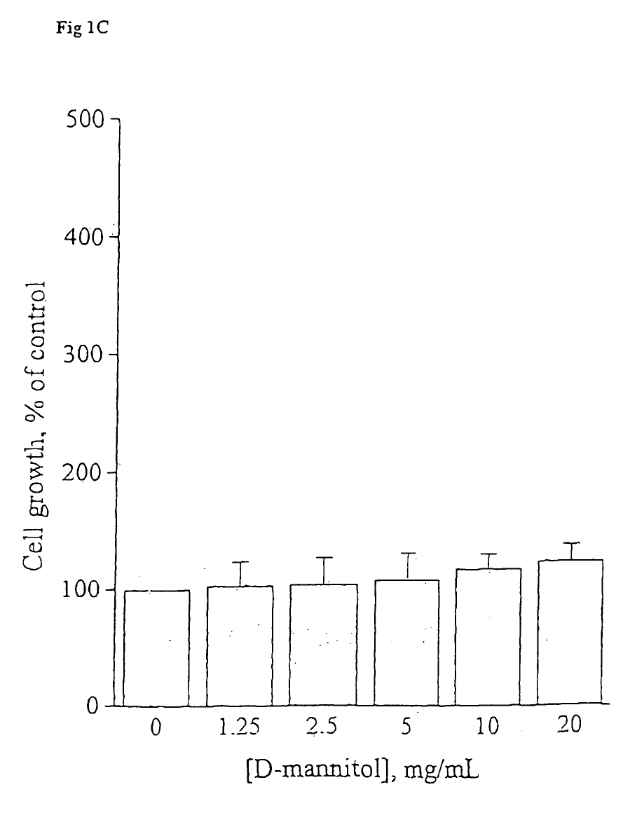

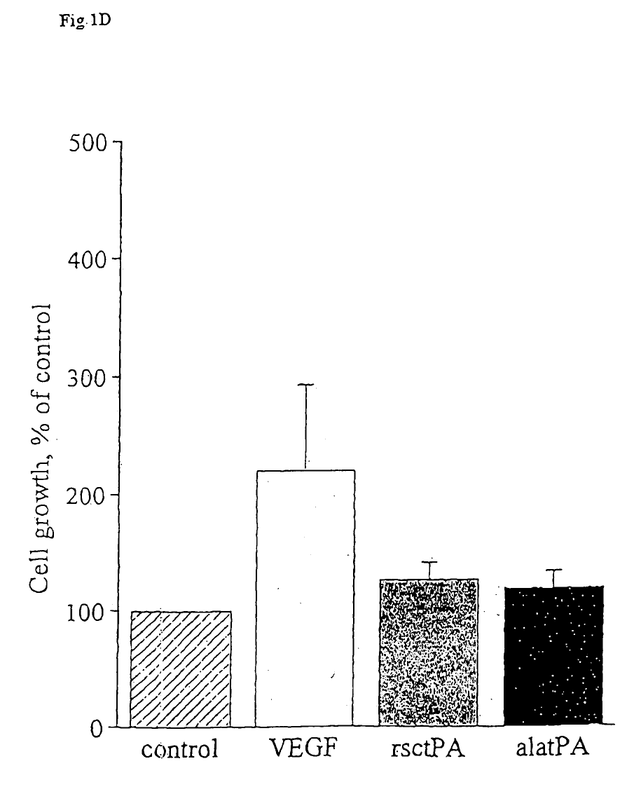

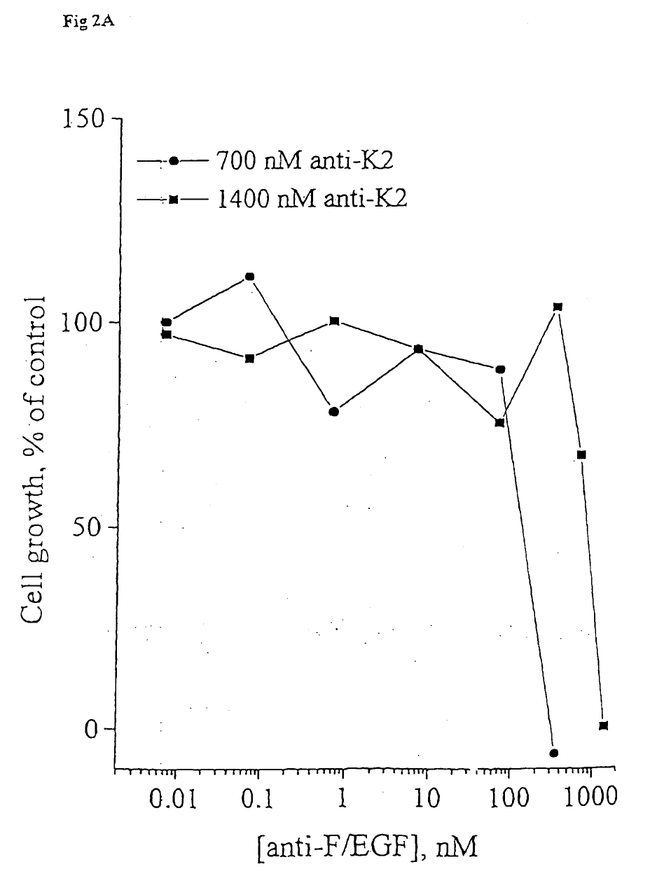

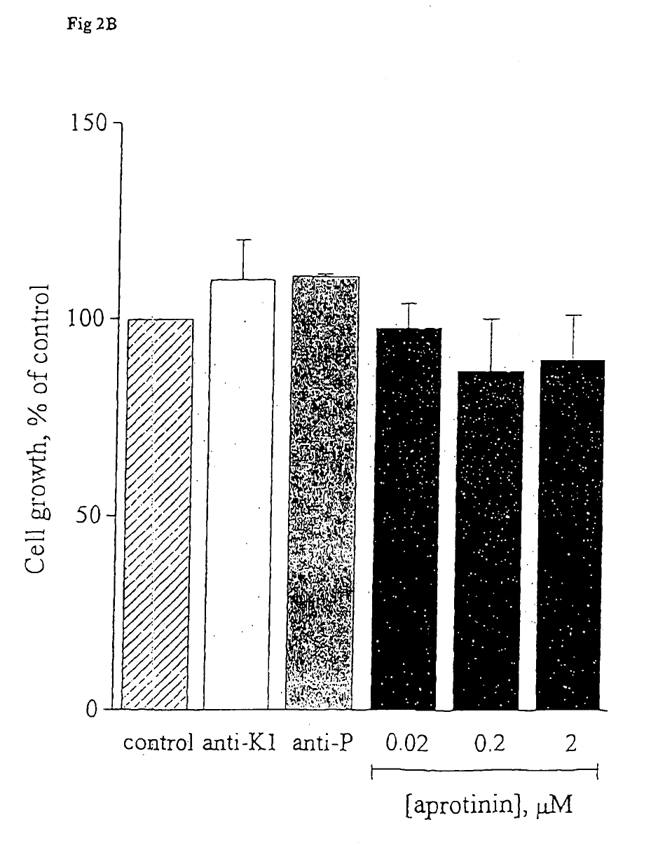

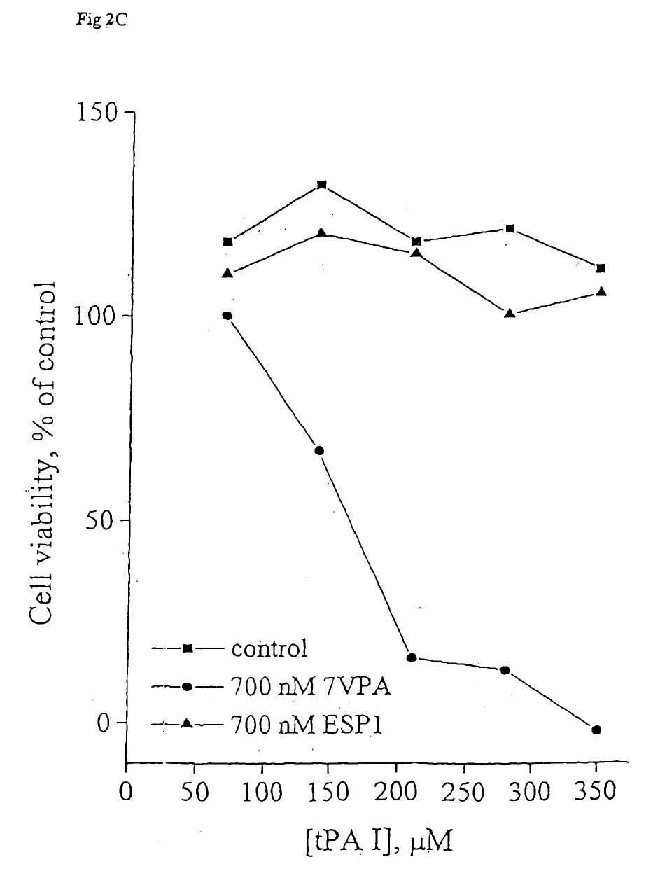

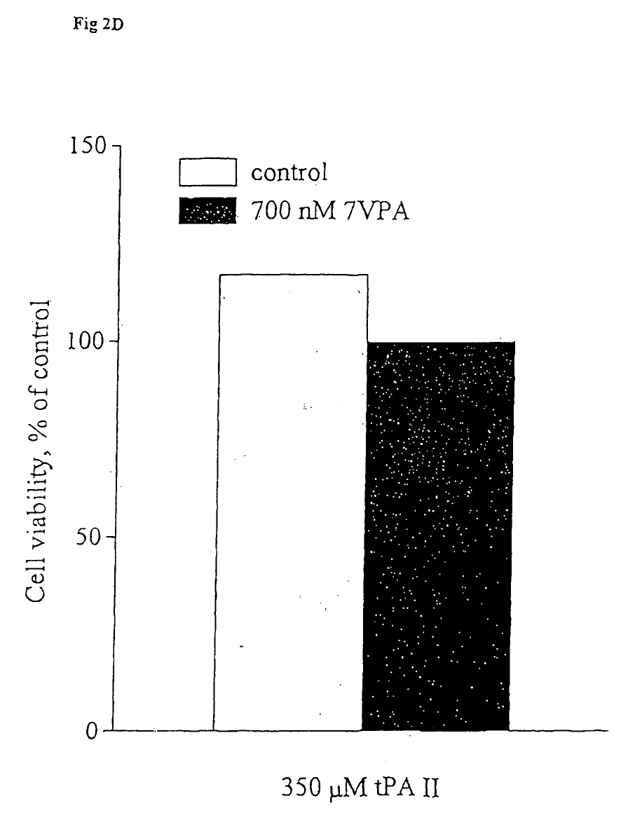

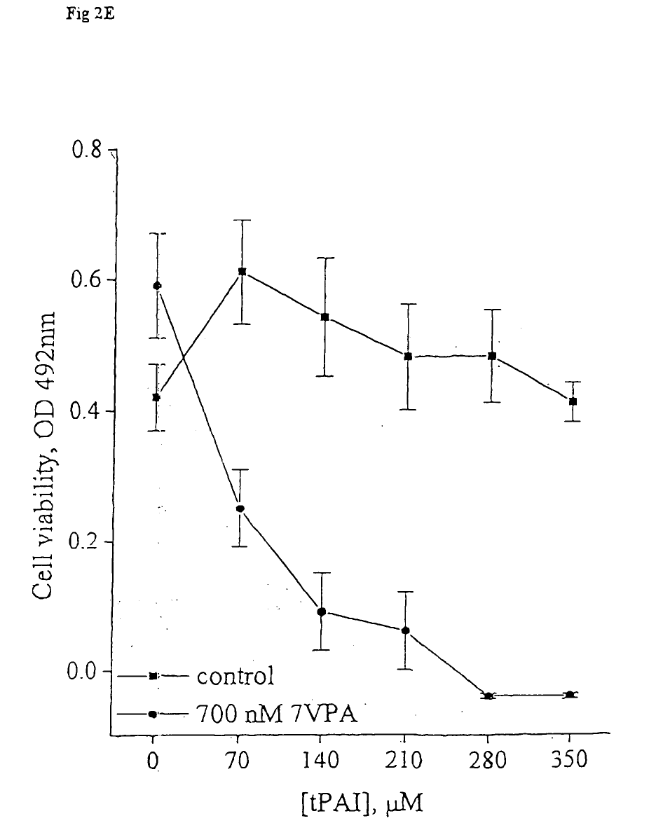

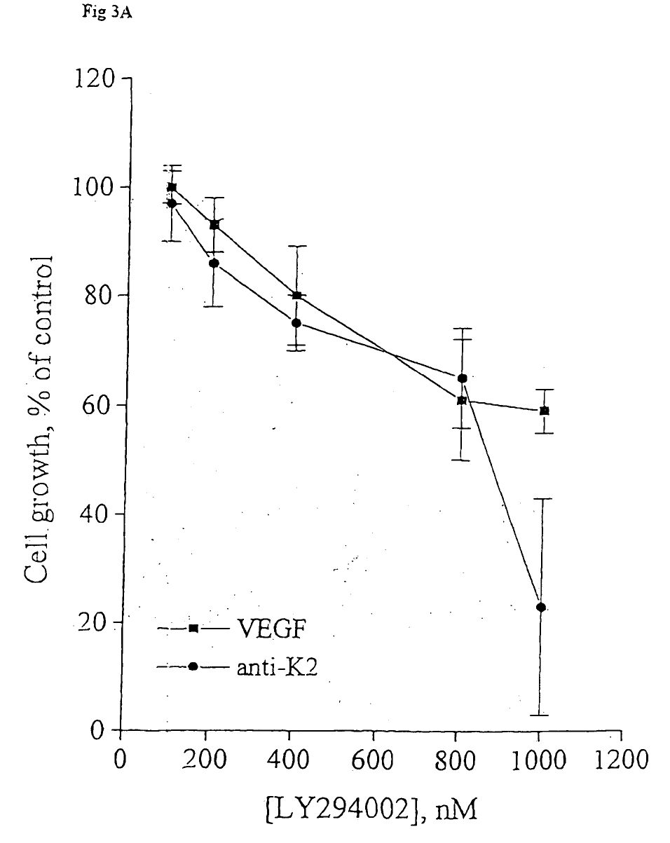

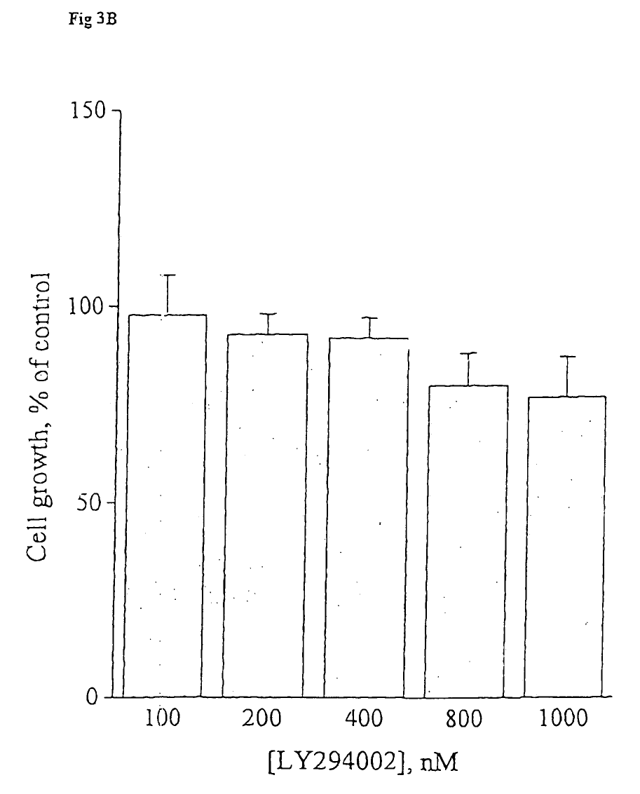

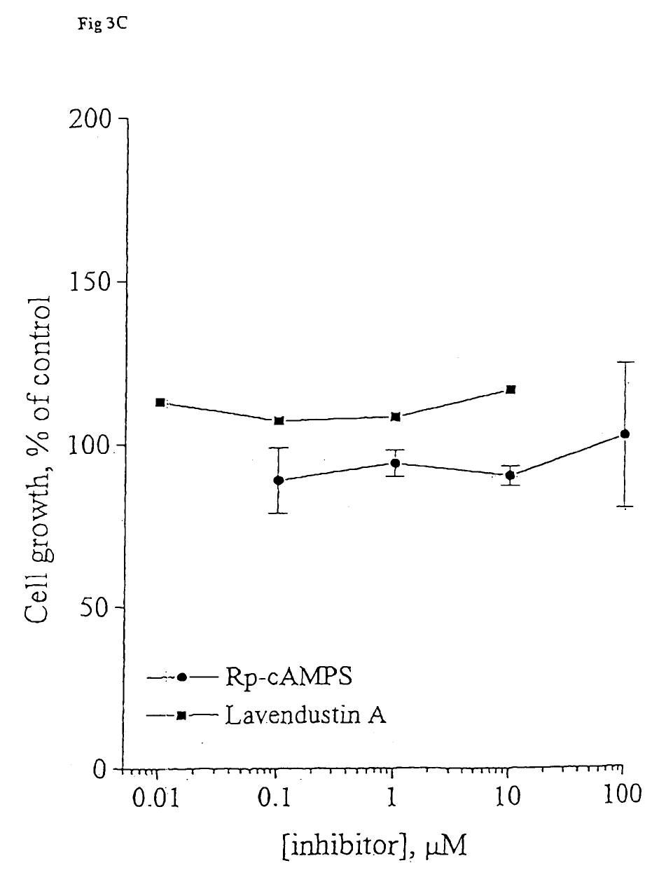

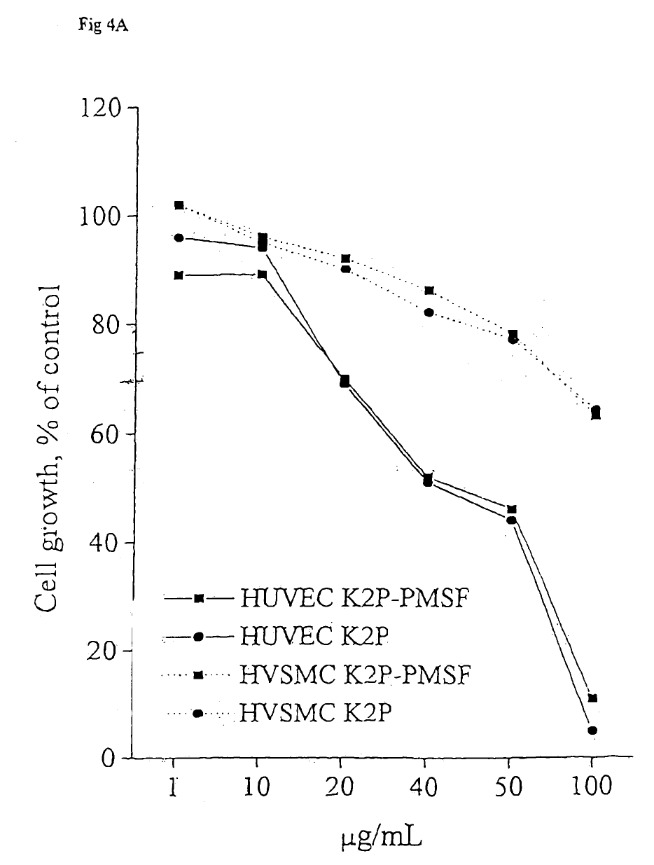

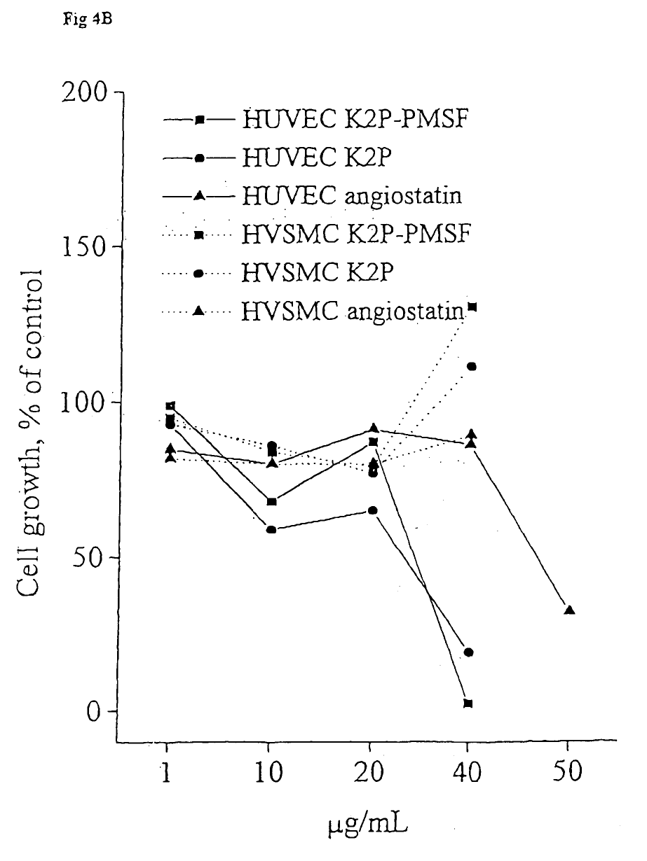

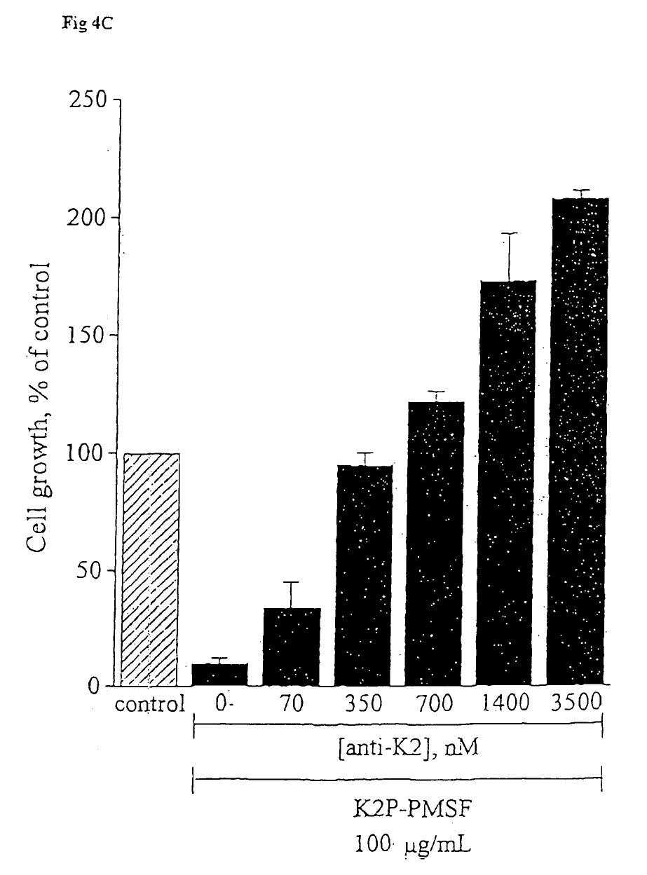

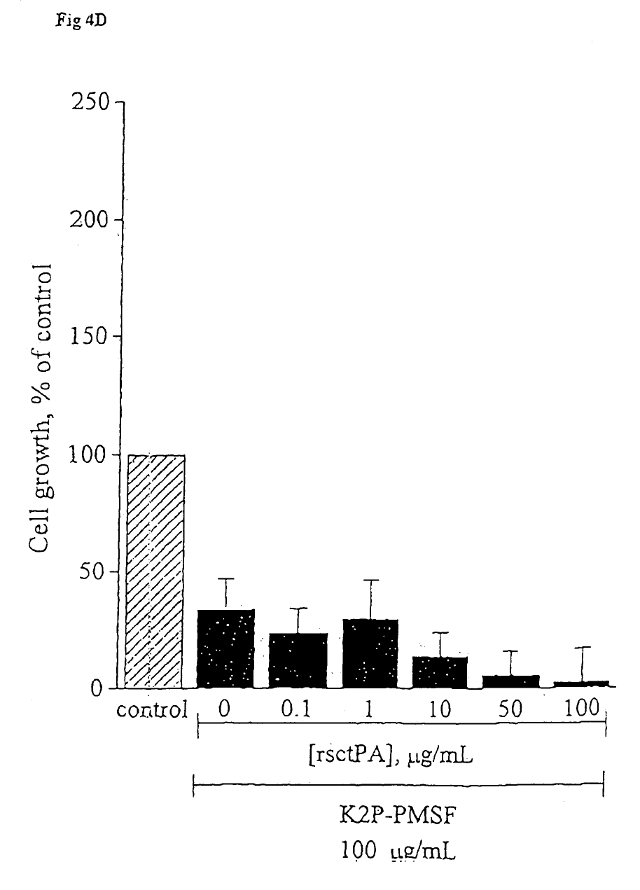

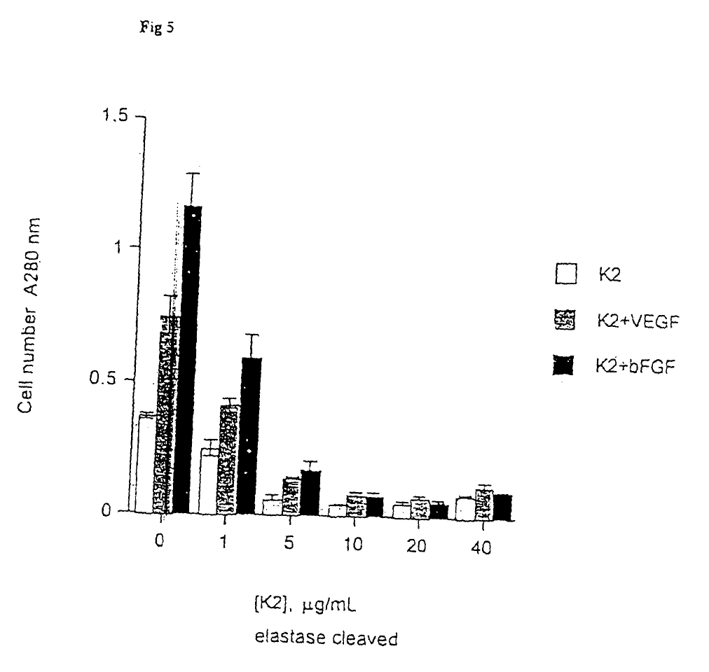

Each batch of HUVEC and HVSMC used in this study was shown to secrete tPA at levels shown previously (4.6 and 4.9 ng/10⁵cells/24 h respectively). In order to identify possible effects of the different modules of endogenously secreted tPA on EC growth, HUVEC were incubated with a panel of monoclonal antibodies directed against the individual domains of tPA in the presence of 2% FBS but no other EC growth factors. An antibody that recognized the kringle 2 (K2) domain of tPA (7VPA) (Wojta J et al, 1989) caused a dose dependent increase in EC proliferation as determined colorimetrically (FIGS. 1A and 1B). A four fold increase in HUVEC growth was observed at 500 μg/mL (3.5 μM) the highest concentration of antibody used which was statistically significant as compared with the lowest concentration of antibody used (p<0.005). Similar results were obtained when cell numbers were counted directly after addition of 7VPA to HUVEC (data not shown). Antibodies directed to the finger/EGF-like, kringle 1 (K1) or the protease domains of tPA did not have similar effects on HUVEC growth. The anti-K2 antibody contained mannitol as an additive and as some low molecular weight sugars such as 2-deoxy-D-ribose are known to stimulate EC growth mannitol was added directly to HUVEC. No effect of mannitol on EC growth was observed at similar concentrations to those contained in the antibody (FIG. 1C) indicating that mannitol was not responsible for the increase in HUVEC proliferation. In addition, the increase in EC growth was maintained following dialysis of the anti-K2 antibody excluding the possibility that low molecular weight agents mediated EC proliferation observed (data not shown). Anti-K2 induced HUVEC proliferation was specific for EC cultures as no effect of the antibody was observed on HVSMC even though these cells also secrete endogenous tPA (data not shown).

No effect of Exogenous tPA on EC Proliferation

Potential explanations for stimulation of EC growth as a consequence of binding the kringle 2 domain of tPA were explored further. In order to investigate whether exogenously added tPA could also mediate HUVEC growth, recombinant single chain tPA (rsctPA) or a mutant catalytically inactive tPA (alatPA) were added to HUVEC cultures. At physiological concentrations (10 ng/mL) neither rsctPA nor alatPA could stimulate EC growth as compared with vascular endothelial growth factor (VEGF) (FIG. 1D) or anti-kringle 2 antibody. This indicates that tPA alone was not sufficient to stimulate HUVEC growth, and therefore possible release of tPA from EC storage sites by the antibody was not responsible for the EC proliferation observed.

Role of Finger Domain of tPA in Mediating EC Proliferation

Stimulation of EC growth by blocking the kringle 2 domain of tPA may have been due to a activation of another part of the tPA molecule, perhaps resulting from a conformational change in tPA. To identify whether a second domain of tPA mediated the increase in HUVEC proliferation following kringle 2 binding, HUVEC cultures were stimulated to proliferate with anti-K2 antibody with the simultaneous addition of antibodies directed to finger/EGF-like, kringle 1 or protease domains of tPA. An anti-finger/EGF monoclonal dose-dependently inhibited HUVEC proliferation induced by anti-K2 antibody (FIG. 2A). Neither an anti-K1 antibody nor an antibody that inhibited the, catalytic activity of tPA could block the increase in cell growth. In addition, the plasmin inhibitor, aprotinin could not block anti-kringle 2 induced EC proliferation (FIG. 2B). These data suggest that following binding K2, tPA mediated EC growth was not dependent on plasmin generation, but on a region of tPA located within the finger or EGF-like domains. To identify this region further a peptide was synthesized that corresponded to residues 7-17 of the finger domain (tPA I). This peptide was shown previously to block tPA binding to HUVEC indicating that this region of the finger domain was involved in EC binding. HUVEC were stimulated with 700 nM 7VPA with various concentrations of tPA I for 4 days (FIG. 2C). TPA I had no effect on HUVEC when incubated alone, but dose-dependently blocked 7VPA stimulated EC growth. A control peptide (tPA II) that corresponded to residues 15-26 of the finger domain and that was previously shown not to be involved in tPA binding was incubated with HUVEC. No effect of tPA II was observed on HUVEC growth either in the absence or presence of 7VPA (FIG. 2D). These results indicate that part of the finger domain contained within residues 7-17 mediated EC proliferation following 7VPA binding of the kringle 2 domain. HUVEC had detached and rounded up as early as 24 h following addition of 7VPA and tPA I (data not shown) and HUVEC viability was significantly decreased (FIG. 2E).

Cell Signaling Pathways Downstream of the Finger Domain

We sought to identify the cell signaling pathway that might be responsible for the increased cell proliferation following 7VPA stimulation. We used a protein kinase A inhibitor (Rp-cAMPS), an EGF receptor tyrosine kinase inhibitor (Lavendustin A) and a PI3-kinase inhibitor (LY294002). Rp-cAMPS was previously shown to block canine cell growth stimulated with tPA. Lavendustin A and LY294002 blocks the VEGF stimulated pathway in EC. FIG. 3A and FIG. 3B shows that at non-toxic doses LY294002 blocked the increase in EC growth mediated by 7VPA as the positive control VEGF stimulated proliferation. Neither the PKA inhibitor nor the EGF receptor tyrosine kinase inhibitor could inhibit 7VPA induced HUVEC growth (FIG. 3C).

Effect of Recombinant Kringle 2 Domain on EC Growth

We have shown that a region located within residues 7-17 of the finger domain of tPA is responsible for mediating HUVEC proliferation observed in this study. Proliferation only occurred in the presence of a specific antibody that bound the K2 domain of tPA. To investigate whether the K2 domain was itself inhibitory, a tPA mutant was obtained that consisted of only the kringle 2 and protease domains (K2P). In order to analyse the effects of the K2 module alone on EC growth, the protease domain was inactivated with PMSF. FIG. 4A shows that both the proteolytically inactivated K2P (K2P-PMSF) and the control K2P fragments inhibited basal EC growth. Toxicity was observed at higher concentrations with both inactivated and control K2P (>40 μg/mL). Marginal toxicity was also observed with basal HVSMC growth which was reduced to 65% of control at the highest concentration used (100 μg/mL). At lower non-toxic doses of K2P and K2-PMSF (<40 μg/mL), bFGF induced EC growth was completely inhibited (FIG. 4B). No inhibition was observed on HVSMC growth in the presence of bFGF. These data indicate EC specificity. The activity of K2P and K2P-PMSF was comparable to angiostatin. 50% inhibition was observed at 30 μg/mL for K2P and K2P-PMSF and 45 μg/mL for angiostatin.

The fact that both K2P-PMSF and K2P had similar effects on EC indicates that inhibition observed was unlikely to be due to alterations in K2P that resulted from the PMSF inactivation process. Similarly as no differences between the catalytic and catalytically inactivated fragments were found indicates that inhibition so was not due to excessive proteolytic activity of K2P, but rather due to a region located within the K2 domain. To confirm that EC growth inhibition observed was due to the K2 domain, HUVEC were incubated with K2P-PMSF and increasing concentrations of anti-K2 antibody (7VPA). FIG. 4C shows that anti-K2 could dose-dependently recover the growth inhibition induced by K2P-PMSF whereas tPA could not reverse inhibition (FIG. 4D). 700 nM anti-kringle 2 antibody stimulates cell growth two fold without the presence of the kringle 2 fragment, with the K2P-PMSF growth was returned to baseline levels at 700 nM anti-K2.

### EXAMPLE 2

### Isolation of Kringle 2 Domain

We have obtained highly pure preparations of kringle 2 by two methods:

(i) The fragment K2P consisting of the kringle 2 domain and the protease domain was subjected to limited digestion with elastase. The resulting digest was applied to a lysine-Sepharose column and the absorbed kringle 2 was specifically eluted with 0.2 M ε-amino-n-caproic acid (EACA).

(ii) We have made a recombinant kringle 2 domain using the Pichia pastoris yeast expression system.

Materials and Methods

Purification of the Kringle 2 Domain of tPA from K2P

17.4 mg of tPA consisting of the kringle 2 domain and the protease domain (K2P) was incubated with 150 μg elastase for 3 h at room temperature with shaking. The reaction was terminated with 2 mM PMSF and incubated for a further 30 min at room temperature. The digest fleas extensively dialysed against 50 mM Tris-HCl, 50 mM, NaCl pH 7.7 and was applied to a lysine-Sepharose column previously equilibrated with at least three bed volumes of binding buffer (50 mM Tris-HCl, 50 mM NaCl pH 7.7). The column was wasted with binding buffer until absorbance at 280 nm was less than 0.005 nm. The adsorbed kringle 2 domain was eluted with 0.2 M EACA.

Expression and Purification of Recombinant K2

The kringle 2 domain was expressed in the Pichia pastoris yeast expression system. For this purpose the plasmid pPIC9K containing the K1_{Pg}-IEGR-K2_{tPA} coding sequence, was used. This construct consisted of the kringle 1 of plasminogen separated by a factor Xa (FXa) cleavage site (IEGR) followed by the kringle 2 domain of tPA KM71 cells transformed with the construct were streaked on YPD plates. Single colonies were picked and used to inoculate 100 mL buffered minimal glycerol-complex (BMGY) medium in 1 L culture flasks. Cells were growth at 30° C. until an A₆₀₀ of between 2-6 was reached in a shaking incubator. The cells were harvested by centrifugation at 3000 g for 5 min at room temperature. The supernatant was decanted and the pellet was re-suspended in 50 mL of buffered minimal methanol-comples (BMMY) medium to induce expression of the K1_{Pg}-IEGR-K2_{tPA} containing construct. Methanol (100%) was added every 24 h to a final concentration of 0.5% (v/v). The cell conditioned media were collected 48 h after methanol induction. Purification of the fusion polypeptide K1_{Pg}-IEGR-K2_{tPA} was achieved by affinity chromatography. Yeast supernatant was concentrated in a Centriprep centrifugal filter, dialysed against column binding buffer (50 mM Tris HCl/50 mM NaCl, pH 7.7) and loaded onto a column of lysine-Sepharose equilibrated with the same buffer. The column was washed with at least five bed volumes of binding buffer and the adsorbed fusion protein eluted with binding buffer containing 0.2 M EACA. The purified fusion proteins were then dialysed against 50 mM Tris-HCl/50 nM NaCl, pH 7.7. The two kringle domains were separated by cleavage with factor Xa. K1_{Pg}-IEGR-K2_{tPA} was incubated with FXa at a ratio of peptide:enzyme 50:1 and incubated for 3 h at 37° C. The digest was applied to a lysine-Sepharose column and washed with 50 mM Tris-HCl/50 mM NaCl, pH 7.7. After baseline absorbance decreased to <0.005, a gradient of EACA was applied in order to resolve the two kringles by virtue of their differential lysine binding capacities.

Results

We have obtained highly purified preparations of the kringle 2 domain. Kringle 2 was obtained by limited digestion with elastase. The kringle 2 domain migrates with a molecular mass of approximately 12 kDa. A Western blot confirmed the reactivity of the kringle 2 domain with both a polyclonal anti-tPA antibody and a monoclonal antibody against the anti-kringle 2 domain of tPA.

Kringle 2 was also expressed in yeast. For this a fusion polypeptide containing the coding sequence for the kringle 2 domain of tPA linked to the kringle 1 domain of plasminogen was used. Purified fusion polypeptide migrated at a molecular weight of approximately 26 kDa. Following digestion of the fusion polypeptide with factor Xa, kringle 2 of tPA migrated at a molecular mass of 12-13 kDa as expected whereas kringle 1 of plasminogen migrated at a higher than expected molecular weight of approximately 17-18 kDa. The digest was loaded on to a lysine-Sepharose column and the two kringles were resolved by the virtue of their differential lysine binding capabilities. Kringle 2 containing fractions were eluted. A Western blot was carried out with a polyclonal anti-tPA antibody. These data confirm that the lower 12-13 kDa band is the kringle 2 domain of tPA. Only the lower kringle 2 band of tPA was detected in the lane which contained the digested polypeptide with factor Xa enzyme.

The elastase cleaved kringle 2 was tested for its ability to inhibit endothelial cell proliferation. FIG. 5 shows kringle 2 significantly inhibited both basal human umbilical vein endothelial cell (HUVEC) growth and basis fibroblast growth factor (bFGF) and vascular endothelial growth factor (VEGF) stimulated growth. These data confirm our earlier work that the kringle 2 domain of tPA is both cytotoxic and anti-proliferative to endothelial cells.

1. A compound which inhibits binding of kringle 2 domain of tissue plasminogen activator (tPA) to endothelial cells for use in stimulating proliferation of endothelial cells.

2. A compound according to claim 1 which comprises an anti-kringle 2 domain antibody.

3. In combination, (a) a compound as defined in claim 1 or 2, and (b) tPA or a fragment thereof comprising the finger domain or a variant of either thereof for simultaneous or sequential administration for use in stimulating proliferation of endothelial cells.

4. A compound or combination according to any one of claims 1, 2 or 3 for use in wound healing, coronary artery disease and critical limb ischaemia.

5. A method for the identification of a substance useful for stimulating proliferation of endothelial cells, comprising incubating a kringle 2 domain of tPA with an endothelial cell membrane in the presence of a test substance, monitoring for binding of the kringle 2 domain to the endothelial cell membrane, and determining thereby whether the test substance is useful in stimulating proliferation of endothelial cells.

6. A method according to claim 5, further comprising formulating a test substance identified as stimulating proliferation of endothelial cells with a pharmaceutically acceptable carrier.

7. A kringle 2 domain of tPA or a variant thereof for use in reducing endothelial cell proliferation or inducing cell death.

8. In combination, a compound as defined in claim 1 or 2 and a compound which inhibits binding of the finger domain of tPA to endothelial cells, for simultaneous or sequential administration for use in reducing endothelial cell proliferation or inducing cell death.

9. A compound or combination according to claim 7 or 3 for use in the treatment of solid tumours, rheumatoid arthritis and diabetic retinopathy.

10. A polynucleotide encoding a kringle 2 domain of tPA and comprising SEQ ID NO: 2 or a variant thereof for use in reducing endothelial cell proliferation or inducing cell death.

11. An expression vector comprising a polynucleotide as defined in claim 9 for use in reducing endothelial cell proliferation or inducing cell death.

12. A polynucleotide or expression vector according to claim 9 or claim for use in the treatment of solid tumours, rheumatoid arthritis and diabetic retinopathy.

13. A method of stimulating proliferation of endothelial cells comprising contacting said cells with a compound which inhibits binding of kringle 2 domain of tPA to endothelial cells.

14. A method according to claim 13, further comprising contacting said cells with tPA or a fragment thereof comprising the finger domain of tPA or a variant of either thereof.

15. A method of reducing endothelial cell proliferation or inducing cell death comprising contacting said cell with a kringle 2 domain of tPA or a variant thereof.

16. A method of reducing endothelial cell proliferation or inducing cell death comprising contacting said cell with a compound as defined in claim 1 or 2 and a compound which inhibits binding of the finger domain of tPA to endothelial cells.


SEQUENCE LISTING (2 sequences)

SEQ ID NO: 1 - protein, 527 aa, Homo sapiens
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 2 - DNA, 246 bp, Homo sapiens
tgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgc